(19) United States
(12) Patent Application Publication
(10) Pub. No.: US 2020/0283531 A1
(43) Pub. Date: Sep. 10, 2020

(54) Lipid-Based Antigens and T-Cell Receptors on NK Cells

(71) Applicant: NantCell, Inc., Culver City, CA (US)

(72) Inventors: Kayvan Niazi, Culver City, CA (US); Marcos Sixto, Culver City, CA (US); Annie Shin, Culver City, CA (US); Phil Liu, Culver City, CA (US)

(21) Appl. No.: 16/753,330

(22) PCT Filed: Oct. 4, 2018
(86) PCT No.: PCT/US2018/054418

§ 371 Date: Apr. 2, 2020

Related U.S. Application Data
(60) Provisional application No. 62/568,785, filed on Oct. 5, 2017.

(51) Int. Cl. C07K 16/28; A61K 35/17; C12N 5/0783; A61K 39/39; A61K 9/00

(52) U.S. Cl. CPC C07K 16/2833; A61K 35/17; C12N 5/0638; C12N 2510/00; A61K 9/0019; C07K 2317/622; A61K 39/39

(57) ABSTRACT

Compositions, methods and uses of genetically modified NK cells to elicit immune response against cells infected with microorganisms are presented. In some embodiments, NK cells can be genetically modified with a recombinant nucleic acid that includes a segment encoding an extracellular single-chain variant fragment that specifically binds a CD1-lipid antigen complex and another segment encoding an intracellular activation domain, and a linker between those segments. In other embodiments, the NK cells can be genetically modified with a recombinant nucleic acid that includes a segment encoding an a chain T cell receptor and a β chain T cell receptor, and another segment encoding at least a portion of CD3δ and at least a portion of CD3γ. The genetically modified NK cells can be administered to the patient infected with microorganism to trigger immune response specific to the cells infected with the microorganism.

This application claims priority to our co-pending WIPO Application having the serial number PCT/US2018/054418, filed Oct. 4, 2018 and US Provisional Application having the Ser. No. 62/568785, filed Oct. 5, 2017, which is incorporated in its entirety herein.

### FIELD OF THE INVENTION

The field of the invention is immunotherapy, especially as it relates to modified NK cells that express a chimeric T cell receptor that specifically recognizes complexes of lipid antigens generated by microorganisms with specific CD1 proteins.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Invasion of foreign pathogens (e.g., bacteria, etc.) into a host organism typically triggers presentation of various lipid antigens from the foreign pathogens, especially those specific and/or unique to the foreign pathogens, on the host's antigen presenting cell surface via a CD1 receptor. More specifically, lipid antigens are processed intracellularly and will bind to one of the CD1 isoforms (CD1a, b, c, and d) in the endosome, which then are transported to the cell surface. The lipid antigen-CD1 receptor complex on the cell surface then interacts with a T cell receptor of a T cell, and triggers a T-cell mediated immune response against the cells infected by the foreign pathogens.

The T cell receptor includes two highly variable chains (e.g., alpha and β chains) that are responsible for recognizing antigens presented on the cell surface. Similar to immunoglobulins, hypervariability (and therefore specificity) of the T cell receptor chain is determined by somatic genetic recombination of the DNA. More recently, genetically engineered receptors, chimeric antigen receptors (CARs), have been developed by grafting antigen specific binding portions onto signaling portions to so drive immune cells carrying the CAR to the targeted cells (e.g., infected cells, cancer cells, etc.). Notably, however, such approach has traditionally been used in the context of MHC-I and MHC-II presented antigens.

Thus, even though some mechanisms of lipid antigen presentation from certain pathogens and various methods of targeting specific cells using genetically engineered receptors are known, modulation of the innate immune system to specifically target cells presenting lipid antigen of interest have remained largely unexplored. Thus, there remains a need for improved methods and uses to use antigen specificity of T cells or antibodies to modify NK cells to specifically attack cells affected by pathogens of interest.

### SUMMARY OF THE INVENTION

The inventive subject matter is directed to various compositions of, methods for, and use of genetically modified immune competent cells that express chimeric protein comprising an extracellular domain that specifically recognizes a CD1-lipid antigen complex and further comprising an activation domain that triggers an immune response of NK cells against the cells presenting the CD1-lipid antigen complex.

Thus, one aspect of the subject matter includes a recombinant nucleic acid that can be transcribed in the NK cells. The recombinant nucleic acid includes a first nucleic acid segment encoding an extracellular single-chain variant fragment that specifically binds a CD1-lipid antigen complex, and a second nucleic acid segment encoding an intracellular activation domain. The first and second nucleic acid segments are coupled with a third nucleic acid segment encoding a linker between the extracellular single-chain variant fragment and the intracellular activation domain. Preferably, the first, second, and third segments are arranged such that the extracellular single-chain variant fragment, the intracellular activation domain, and the linker form a single chimeric polypeptide. In further aspects, the recombinant nucleic acid is an mRNA that may encode at least a TCR alpha and TCR beta chain, and that may additionally also encode CD3delta and CD3 gamma.

Preferably, the extracellular single-chain variant fragment comprises a V_{L} domain and a V_{H} domain of a monoclonal antibody against the CD1-lipid antigen complex. In such embodiment, it is also preferred that the recombinant nucleic acid further comprises a spacer between the V_{L} domain and the V_{H} domain.

In some embodiments, the CD1-lipid antigen complex comprises at least one of the following: CD1a, CD1b, CD1c. In other embodiments, the CD1-lipid antigen complex comprises at least one of the following: mycobacterial phospholipids, glycolipids, mycolic acids, lipopeptides, mycoketides, and isoprenoids. In such embodiments, it is contemplated that CD1-lipid antigen complex may comprise a lipid antigen of M. Tuberculosis. Further, where the CD1-lipid antigen complex may comprise a lipid antigen of M. Tuberculosis, the lipid antigen of M. Tuberculosis can be a mycolic acid.

Preferably, the intracellular activation domain comprises an immunoreceptor tyrosine-based activation motif (ITAM) that triggers ITAM-mediated signaling in a natural killer cell. In some embodiments, the intracellular activation domain comprises a portion of CD3ζ and/or a portion of CD28 activation domain. Also preferably, the linker comprises a CD28 transmembrane domain or a CD3ζ transmembrane domain.

In another aspect of the inventive subject matter, the inventors contemplate a method for inducing an NK cell immune response in a patient infected with a mycolic acid producing microorganism. In this method, a genetically modified NK cell expressing a recombinant protein is provided. Most typically, the genetically modified NK cell is selected or derived from the group consisting of: aNK, haNK, and taNK. The recombinant protein has an extracellular single-chain variant fragment that specifically binds a CD1-lipid antigen complex and an intracellular activation domain. Additionally, the extracellular single-chain variant fragment and the intracellular activation domain are coupled with a transmembrane linker. The method continues with administering the genetically modified NK cell to the patient in a dose and a schedule effective to reduce a number of cells infected with the microorganism in the patient and/or to reduce the number of microorganisms in the patient. Typically, the administering the genetically modified NK cell is performed by intravenous injection.

Preferably, the extracellular single-chain variant fragment comprises a V_{L} domain and a V_{H} domain of a monoclonal antibody against the CD1-lipid antigen complex. In some embodiments, the extracellular single-chain variant fragment further comprises a spacer between the V_{L} domain and the V_{H} domain. In some embodiments, the CD1-lipid antigen complex comprises at least one of the following: CD1a, CD1b, CD1c. In other embodiments, the CD1-lipid antigen complex comprises at least one of the following: mycobacterial phospholipids, glycolipids, mycolic acids, lipopeptides, mycoketides, and isoprenoids. In such embodiments, it is contemplated that CD1-lipid antigen complex may comprise a lipid antigen of M. Tuberculosis. Further, where the CD1-lipid antigen complex may comprise a lipid antigen of M. Tuberculosis, the lipid antigen of M. Tuberculosis can be a mycolic acid.

Preferably, the intracellular activation domain comprises an immunoreceptor tyrosine-based activation motif (ITAM) that triggers ITAM-mediated signaling in a natural killer cell. In some embodiments, the intracellular activation domain comprises a portion of CD3ζ and/or a portion of CD28 activation domain. Also preferably, the linker comprises a CD28 transmembrane domain or a CD3ζ transinembrane domain.

Still another aspect of inventive subject matter includes a recombinant nucleic acid composition that can be transcribed and/or translated in the NK cells. The recombinant nucleic acid includes a first nucleic acid segment encoding an α chain T cell receptor and a β chain T cell receptor, which are separated by a first self-cleaving 2A peptide sequence. Preferably, at least one of the α chain T cell receptor and the β chain T cell receptor together specifically bind a CD1-lipid antigen complex. The recombinant nucleic acid may also include a second nucleic acid segment encoding at least a portion of CD3ζ and at least a portion of CD3γ, which may be separated by a second self-cleaving 2A peptide sequence. In some embodiments, the first nucleic acid segment and the second nucleic acid segment are separated by a third self-cleaving 2A peptide sequence.

Preferably, the portion of CD3γ comprises an immunoreceptor tyrosine-based activation motif (ITAM), and/or the portion of CD3δ comprises an immunoreceptor tyrosine-based activation motif (ITAM). In some embodiments, the CD1-lipid antigen complex comprises at least one of the following: CD1a, CD1b, CD1c. In other embodiments, the CD1-lipid antigen complex comprises at least one of the following: mycobacterial phospholipids, glycolipids, mycolic acids, lipopeptides, mycoketides, and isoprenoids. In such embodiments, it is contemplated that CD1-lipid antigen complex may comprise a lipid antigen of M. Tuberculosis. Further, where the CD1-lipid antigen complex may comprise a lipid antigen of M. Tuberculosis, the lipid antigen of M. Tuberculosis can be a mycolic acid.

In still another aspect of the inventive subject matter a genetically modified cytotoxic cell is contemplated that is preferably a genetically modified NK cell. The cytotoxic cells include a recombinant nucleic acid encoding a chimeric protein having 1) an extracellular single-chain variant fragment that specifically binds a CD1-lipid antigen complex, 2) an intracellular activation domain, and 3) a transmembrane linker coupling the extracellular single-chain variant fragment to the intracellular activation domain.

Preferably, the extracellular single-chain variant fragment comprises a V_{L} domain and a V_{H} domain of a monoclonal antibody against the CD1-lipid antigen complex. In some embodiments, the recombinant nucleic acid further comprises a spacer between the V_{L} domain and the V_{H} domain.

In some embodiments, the CD1-lipid antigen complex comprises at least one of the following: CD1a, CD1b, CD1c. In other embodiments, the CD1-lipid antigen complex comprises at least one of the following: mycobacterial phospholipids, glycolipids, mycolic acids, lipopeptides, mycoketides, and isoprenoids. In such embodiments, it is contemplated that CD1-lipid antigen complex may comprise a lipid antigen of M. Tuberculosis. Further, where the CD1-lipid antigen complex may comprise a lipid antigen of M. Tuberculosis, the lipid antigen of M. Tuberculosis can be a mycolic acid.

In some embodiments, the intracellular activation domain comprises an immunoreceptor tyrosine-based activation motif (ITAM) that triggers ITAM-mediated signaling in a natural killer cell. In other embodiments, the intracellular activation domain comprises a portion of CD3ζ and/or a portion of CD28 activation domain. Further, in some embodiments, the linker comprises a CD28 transmembrane domain or a CD3ζ transmembrane domain.

In yet another aspect of the inventive subject matter a genetically modified cytotoxic cell is contemplated that includes a recombinant nucleic acid encoding a protein complex having α chain T cell receptor, a β chain T cell receptor, at least a portion of CD3δ, and at least a portion of CD3γ. Preferably, the first nucleic acid segment and the second nucleic acid segment are separated by a third self-cleaving 2A peptide sequence.

In some embodiments, the portion of CD3γ and/or the portion of CD3δ comprise an immunoreceptor tyrosine-based activation motif (ITAM). In some embodiments, the CD1-lipid antigen complex comprises at least one of the following: CD1a, CD1b, CD1c. In other embodiments, the CD1-lipid antigen complex comprises at least one of the following: mycobacterial phospholipids, glycolipids, mycolic acids, lipopeptides, mycoketides, and isoprenoids. In such embodiments, it is contemplated that CD1-lipid antigen complex may comprise a lipid antigen of M. Tuberculosis. Further, where the CD1-lipid antigen complex may comprise a lipid antigen of M. Tuberculosis, the lipid antigen of M. Tuberculosis can be a mycolic acid.

In a still further aspect of the inventive subject matter, the inventors contemplate a method for inducing an NK cell immune response in a patient infected with a mycolic acid producing microorganism. In this method, a genetically modified NK cell expressing a protein complex is provided. The protein complex includes at least an α chain T cell receptor, a β chain T cell receptor, at least a portion of CD36, and at least a portion of CD3γ. Typically, the genetically modified NK cell is selected from and/or derived from the group consisting of: aNK, haNK, and taNK. The method further continues with administering the genetically modified NK cell to the patient in a dose and a schedule effective to reduce a number of cells infected with the microorganism in the patient. Typically, the administering the genetically modified NK cell is performed by intravenous injection.

Preferably, the first nucleic acid segment and the second nucleic acid segment are separated by a third self-cleaving 2A peptide sequence. Also preferably, the portion of CD3γ comprises an immunoreceptor tyrosine-based activation motif (ITAM) and/or the portion of CD3δ comprises an immunoreceptor tyrosine-based activation motif (ITAM). In some embodiments, the CD1-lipid antigen complex comprises at least one of the following: CD1a, CD1b, CD1c. In other embodiments, the CD1-lipid antigen complex comprises at least one of the following: mycobacterial phospholipids, glycolipids, mycolic acids, lipopeptides, mycoketides, and isoprenoids. In such embodiments, it is contemplated that CD1-lipid antigen complex may comprise a lipid antigen of M. Tuberculosis. Further, where the CD1-lipid antigen complex may comprise a lipid antigen of M. Tuberculosis, the lipid antigen of M. Tuberculosis can be a mycolic acid.

Additionally, the inventors also contemplate uses of the recombinant nucleic acids and/or genetically modified cytotoxic cells described above for inducing an NK cell immune response in a patient infected with a microorganism.

Various objects, features, aspects and advantages of the inventive subject matter will become more apparent from the following detailed description of preferred embodiments, along with the accompanying drawings.

### BRIEF DESCRIPTION OF THE DRAWING

FIG. 1 illustrates three exemplary embodiments of a recombinant chimeric protein expressed on a cell surface.

FIG. 2 illustrates exemplary embodiments of mRNA constructs encoding a T cell receptor protein complex, and interaction between CD1-lipid antigen complex and the T cell receptor protein complex.

FIG. 3A shows a graph depicting cytotoxicity of NK cells expressing a recombinant chimeric protein of FIG. 1 towards cells presenting a lipid antigen on their surface.

FIG. 3B shows a graph depicting bacterial viability as a function of NK cells expressing the T cell receptor protein complex of FIG. 3.

### DETAILED DESCRIPTION

The inventors have now discovered that cell-mediated cytotoxicity can be effectively and specifically induced against infected cells by genetically modifying an immune competent cell, preferably a cytotoxic immune competent cell (and particularly an NK cell), and administering the so genetically modified cytotoxic cells to a patient that is infected with a microorganism producing a lipid antigen that can be presented by a CD1 molecule. To that end, the inventors further discovered that various recombinant nucleic acid compositions can be generated to so modify the cytotoxic cells (e.g., natural killer (NK) cells) such that the cytotoxic cells can specifically bind to the infected cell that presents a CD1 ligand coupled to a lipid antigen on its surface. Notably, such modified NK cells can act like T-cells, but provide cytotoxicity of an NK cell.

For example, a cytotoxic cell may express a recombinant chimeric protein that has a cytoplasmic tail and transmembrane domain fused with a scFv fragment with selective affinity against CD1 receptor coupled with the lipid antigen. Such genetically modified cytotoxic cell is contemplated to not only exhibit specific recognition to the microorganism-infected cell, but also specific activation upon binding to the infected cell, which presents the lipid antigen on its surface. In additional aspects, genetically modified cytotoxic cells are also contemplated to recognize lipid antigens presented on the host cell surface upon tumorigenesis or development of autoimmunity against the host cell. Upon recognition of the CD1-lipid antigen complex, the activated cytotoxic cells release cytotoxic molecules (e.g., granzyme, perforin, granulysin, etc.) directed against the infected cell, tumor cell, or cells affected by autoimmunity, and ultimately destroy those cells.

As used herein, the term “immune competent cell” refers to a cell that can elicit any type of immune response including, but not limited to, antibody-dependent cell-mediated cytotoxicity, T-cell immune response, humoral immunity, etc. As used herein, the term “bind” refers to, and can be interchangeably used with a term “recognize” and/or “detect”, an interaction between two molecules with a high affinity with a KD of equal or less than 10⁻⁶M, or equal or less than 10⁻⁷M. As used herein, the term “provide” or “providing” refers to and includes any acts of manufacturing, generating, placing, enabling to use, or making ready to use.

Of course, it should be noted that the inventive subject matter is not limited to NK cells, but that all suitable types of immune competent cells are contemplated. Most preferably, the immune competent cells are cytotoxic immune cells including autologous or heterologous NK cells, natural killer T (NKT) cells, a genetically modified NK cells including NK-92 derivatives, which may be modified to have a reduced or abolished expression of at least one killer cell immunoglobulin-like receptor (KIR), which will render such cells constitutively activated (via lack of or reduced inhibition). Therefore, suitable modified cells may have one or more modified killer cell immunoglobulin-like receptors that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated KIR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and/or KIR3DS1. Such modified cells may be prepared, for example, using silencing protocols, CIRSPR-CAS genome editing, or knock-out or knock-down protocols well known in the art. Alternatively, such cells may also be commercially obtained from NantKwest (see URL www.nantkwest.com) as aNK cells (activated natural killer cells). Such cells may then be further modified to express one or more ligands for one or more inhibitory receptors of the NK cells of the host organism.

In addition, the genetically engineered NK cell may also be an NK-92 derivative that is modified to express a high-affinity Fcγ receptor (e.g., CD16, V₁₅₈). Sequences for high-affinity variants of the Fcγ receptor are well known in the art, and all manners of generating and expression are deemed suitable for use herein. Expression of such receptor is believed to allow specific targeting of tumor cells using antibodies that are specific to a patient's cells affected by inflammation (e.g., by autoimmunity, etc.), patient's tumor cells (e.g., neoepitopes), a particular tumor type (e.g., her2neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells (high-affinity natural killer cells). Such cells may then be further modified to express one or more ligands for one or more inhibitory receptors of the NK cells of the host organism.

Further, the genetically engineered NK cell may also be genetically engineered to express a chimeric T-cell receptor. In especially preferred aspects, the chimeric T-cell receptor will have a scFv portion or other ectodomain with binding specificity against an inflammation-associated peptide antigen, a tumor associated peptide antigen, a tumor specific peptide antigen, and a cancer neoepitope. As noted before, there are numerous manners of genetically engineering an NK cell to express such chimeric T-cell receptor, and all manners are deemed suitable for use herein. Alternatively, such cells may also be commercially obtained from NantKwest as taNK cells (‘target-activated natural killer cells’). Such cells may then be further modified to express one or more ligands for one or more inhibitory receptors of the NK cells of the host organism. The inventors contemplates that use of haNK cells or taNK cells may provide dual-specificity of the genetically modified cytotoxic cells as described later to target any cancer cells or autoimmunity-affected cells by recognizing the cancer- or autoimmune-specific epitope and concurrently recognizing the lipid antigen presented on those cell surfaces.

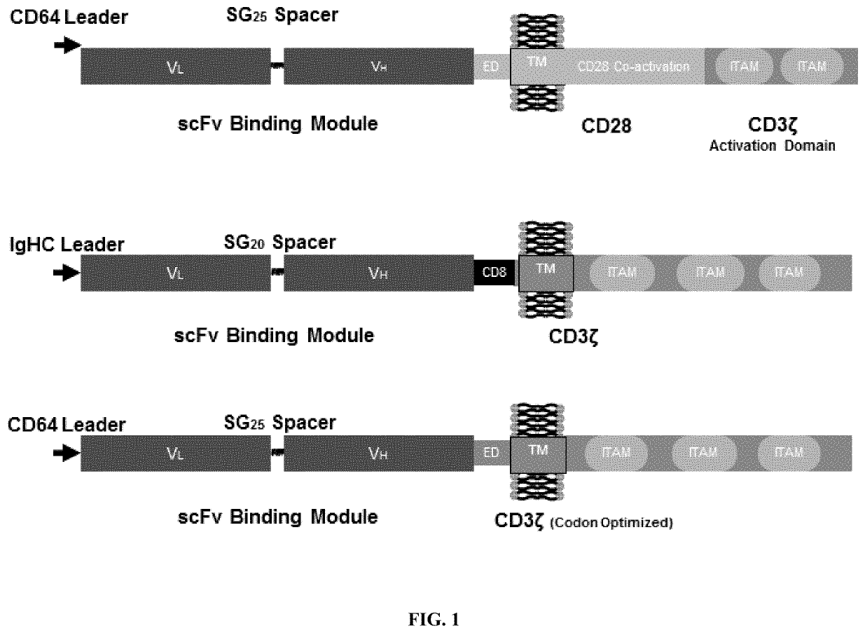

In one preferred aspect of the inventive subject matter, the inventors contemplate that cytotoxic immune competent cells (e.g., NK cells, NKT cells, genetically engineered NK cells (aNK, haNK, taNK, etc), etc.) can be genetically modified to specifically recognize lipid antigens coupled with CD1 molecule by introducing a recombinant nucleic acid composition encoding a recombinant protein to the cytotoxic cells. FIG. 1 schematically shows several exemplary recombinant proteins. Generally, the recombinant protein includes an extracellular single-chain variant fragment, an intracellular activation domain, and a transmembrane linker coupling the extracellular single-chain variant fragment to the intracellular activation domain. Preferably, the recombinant protein is generated from a single chimeric polypeptide translated from a single recombinant nucleic acid. However, it is also contemplated that that the recombinant protein comprises at least two domains that are separately translated from two distinct recombinant nucleic acid such that at least a portion of the recombinant protein can be reversibly coupled with the rest of the recombination protein via a protein-protein interaction motif

Thus, in a preferred embodiment, in which the recombinant protein is encoded by a single recombinant nucleic acid, the recombinant nucleic acid includes at least three nucleic acid segments: a first nucleic acid segment (a sequence element) encoding an extracellular single-chain variant fragment that specifically binds to a CD1-lipid antigen complex; a second nucleic acid segment encoding an intracellular activation domain; and a third nucleic acid segment encoding a linker between the extracellular single-chain variant fragment and the intracellular activation domain.

In this embodiment, the first nucleic acid segment encoding an extracellular single-chain variant fragment includes a nucleic acid sequence encoding a heavy (V_{H}) and light chain (V_{L}) of an immunoglobulin. In a preferred embodiment, the nucleic acid sequence encoding variable regions of the heavy chain (V_{H}) and the nucleic acid sequence encoding variable regions of the light chain (V_{L}) are separated by a linker sequence encoding a short spacer peptide fragment (e.g., at least 10 amino acid, at least 20 amino acid, at least 30 amino acid, etc.). Most typically, the extracellular single-chain variant fragment encoded by the first nucleic acid segment includes one or more nucleic acid sequences that determine the binding affinity and/or specificity to a CD1-lipid antigen complex. Thus, the nucleic acid sequence of VH_{H} and V_{L} can vary depending on the type of CD1 molecule and the lipid antigens the recombinant protein may target to.

Any suitable methods to identify the nucleic acid sequence of V_{H} and V_{L} specific to the CD1-lipid antigen complex are contemplated. For example, a nucleic acid sequence of V_{H} and V_{L} can be identified from a monoclonal antibody sequence database with known specificity and binding affinity to the CD1-lipid antigen complex. Alternatively, the nucleic acid sequence of V_{H} and V_{L} can be identified via an in silico analysis of candidate sequences (e.g., via IgBLAST sequence analysis tool, etc.). In some embodiments, the nucleic acid sequence of V_{H} and V_{L} can be identified via a mass screening of peptides having various affinities to the CD1-lipid antigen complex via any suitable in vitro assays (e.g., flow cytometry, SPR assay, a kinetic exclusion assay, etc.). While it may vary depending on the type of CD1 and lipid antigens, it is preferred that the optimal nucleic acid sequence of V_{H} and V_{L} encodes an extracellular single-chain variant fragment having an affinity to the CD1-lipid antigen complex at least with a K_{D} of at least equal or less than 10⁻⁶M, preferably at least equal or less than 10⁻⁷M, more preferably at least equal or less than 10⁻⁸M. Alternatively, synthetic binders to the CD1-lipid antigen complex may also be obtained by phage panning or RNA display, or by grafting recognition domains from a T cell known to bind a CD1-lipid complex (TCR clone 18 as further described in more detail below).

While it is preferred that that the first nucleic acid segment includes nucleic acid sequence encoding one of each heavy (V_{H}) and light chains (V_{L}), it is also contemplated that in other embodiments, the first nucleic acid segment includes nucleic acid sequence encoding a plurality of heavy (V_{H}) and light chains (V_{L}) (e.g., two heavy (V_{H}) and light chains (V_{L}) for generating a divalent (or even a multivalent) single-chain variable fragments (e.g., tandem single-chain variable fragments). In this embodiment, the sequence encoding one of each heavy (V_{H}) and light chains (V_{L}) can be linearly duplicated (e.g., V_{H}-linker 1-V_{L}-linker 2-V_{H}-linker 3-V_{L}). It is contemplated that the length of the linkers 1, 2, 3 can be substantially similar or same. However, it is also contemplated that the length of linker 2 is substantially different (e.g., longer or shorter) than the length of linker 1 and/or linker 3.

Alternatively, the inventors also contemplate that the extracellular single-chain variant fragment can be substituted with an extracellular domain of T-cell receptor. For example, in some embodiments, the extracellular single-chain variant fragment can be substituted with a portion of α chain and/or β chain of a T cell receptor. In other embodiments, the extracellular single-chain variant fragment can be substituted with a combination of the α chain and β chain. In such embodiment, the nucleic acid sequence of extracellular domain(s) of T-cell receptor, especially hypervariable region(s) of α and β chains can be selected based on the measured, estimated, or expected affinity to the CD1-lipid antigen complex. It is especially preferred that the affinity of extracellular domain of T-cell receptor to the CD1-lipid antigen complex is at least with a K_{D} of at least equal or less than 10⁻⁶ M, preferably at least equal or less than 10⁻⁷ M, more preferably at least equal or less than 10⁻⁸ M.

The recombinant nucleic acid also includes a second nucleic acid segment (a sequence element) encoding an intracellular activation domain of the recombinant protein. Most typically, the intracellular activation domain includes one or more ITAM activation motifs (immunoreceptor tyrosine-based activation motif, YxxL/I-X₆₋₈-YXXL/I), which triggers signaling cascades in the cells expressing the motifs. Any suitable nucleic acid sequences including one or more ITAM activation motifs are contemplated. For example, the sequence of the activation domain can be derived from a cytotoxic cell receptor (e.g., NK cell receptor, NKT cell receptor, etc.) including one or more ITAM activation motif (e.g., intracellular tail domain of killer activation receptors (KARs), NKp30, NKp44, and NKp46, etc.). In another example, the sequence of the activation domain can be derived from a tail portion of a T-cell antigen receptor (e.g., CD3ζ, CD28, etc.). In some embodiments, the nucleic acid sequence of the intracellular activation domain can be modified to add/remove one or more ITAM activation motif to modulate the cytotoxicity of the cells expressing the recombinant protein.

The first and second nucleic acid segments are typically connected via a third nucleic acid segment encoding a linker portion of the recombinant protein. Preferably, the linker portion of the recombinant protein includes at least one transmembrane domain. Additionally, the inventors contemplate that the linker portion of the recombinant protein further includes a short peptide fragment (e.g., spacer with a size of between 1-5 amino acids, or between 3-10 amino acids, or between 8-20 amino acids, or between 10-22 amino acids) between the transmembrane domain and the extracellular single-chain variant fragment, and/or another short peptide fragment between the transmembrane domain and the intracellular activation domain. In some embodiments, the nucleic acid sequence of transmembrane domain and/or one or two short peptide fragment(s) can be derived from the same or different molecule from which the sequence of intracellular activation domain is obtained.

For example, where the intracellular activation domain is a portion of CD3ζ, the entire third nucleic acid segment (encoding both transmembrane domain and short peptide fragment) can be derived from CD3ζ (same molecule) or CD28 (different molecule). In other embodiments, the third nucleic acid segment is a hybrid sequence, in which at least a portion of the segment is derived from a different molecule than the rest of the segment. In a further example, where the intracellular activation domain is a portion of CD3ζ, the sequence of the transmembrane domain can be derived from CD3ζ and a short fragment connecting the transmembrane domain, and the extracellular single-chain variant fragment may be derived from CD28 or CD8.

In still other contemplated embodiments, the recombinant nucleic acid includes a nucleic acid segment encoding a signaling peptide that directs the recombinant protein to the cell surface. Any suitable and/or known signaling peptides are contemplated (e.g., leucine rich motif, etc.). Preferably, the nucleic acid segment encoding an extracellular single-chain variant fragment is located in the upstream of the first nucleic acid segment encoding an extracellular single-chain variant fragment such that the signal sequence can be located in N-terminus of the recombinant protein. However, it is also contemplated that the signaling peptide can be located in the C′ terminus of the recombinant protein, or in the middle of the recombinant protein.

Thus, it should be appreciated that recombinant cytotoxic cells, and especially NK cells can be genetically engineered to express a chimeric antigen receptor in which the extracellular recognition domain will recognize a CD1-lipid antigen complex, and which further includes a transmembrane portion and one or more intracellular activation domains. As will be readily appreciated, such chimeric antigen receptor can be constructed as 1^{st}, 2^{nd}, or 3^{rd} generation CAR and will preferably comprise an scFv domain that specifically recognizes a CD1-lipid complex.

Typically, the recombinant nucleic acid also includes a sequence element that controls expression of the recombinant protein, and all manners of control are deemed suitable for use herein. For example, where the recombinant nucleic acid is an RNA, expression control may be exerted by suitable translation initiation sites (e.g., suitable cap structure, initiation factor binding sites, internal ribosome entry sites, etc.) and a polyA tail (e.g., where length controls stability and/or turnover), while recombinant DNA expression may be controlled via a constitutively active promoter, a tissue specific promoter, or an inducible promoter.

With respect to the CD1-lipid antigen complex, the inventors contemplate that CD1 can be any one of human CD1a, CD1b, CD1c, CD1d isotypes. In addition, any lipid antigens that are generated from a foreign organism (e.g., bacteria, yeast, fungus, mycoplasma, etc.), nutritional substances (e.g., plant food, animal food etc.), or self-lipids generated from a host organism, especially and for example, in an unhealthy condition (e.g., tumor cells, cells affected by autoimmunity, etc.) are contemplated. Such lipid antigens include mycobacterial phospholipids, glycolipids, glycosphingolipids, mycolic acids, lipopeptides, diacylated sulfoglycolipids, mycoketides, isoprenoids, sphingolipids (e.g., aGalCer, sulfatide, iGb3, etc.), glycerolipids (e.g., BbGL-2c, Glc-DAG-s2, LysoPC, cardiolipin, etc.), and lipoprotein. For example, upon infection of a host with mycobacteria (e.g., M. Tuberculosis, having various lipid components in the cell wall), lipid antigens are loaded on one or more isotypes of CD1 (CD1a, CD1b, or CD1c), and such CD1-lipid antigen complex is presented on the infected cell surface. While not all lipid antigens associated with an isotype of CD1 are immunogenic enough to elicit a T-cell mediated immune response, some lipid antigens (e.g., mycolic acids, including alpha-mycolic acid, methoxy-mycolic acid, or keto-mycolic acid, etc.) associated with an isotype of CD1 can effectively elicit T-cell response when the CD1-lipid antigen complex (e.g., CD1b-mycolic acid complex) is presented on the cell surface and recognized by the T cell receptor. In a similar manner, while not all self-lipids are immunogenic, some tumor cells may produce immunogenic lipid antigens (e.g., alpha-galactosylceramide, etc.) that can be loaded on CD1d receptor and presented on the tumor cell surface. CD1d-lipid antigen complex can be recognized by NKT cells, which subsequently trigger release of cytokines against the cells presenting the CD1d-lipid antigen complex.

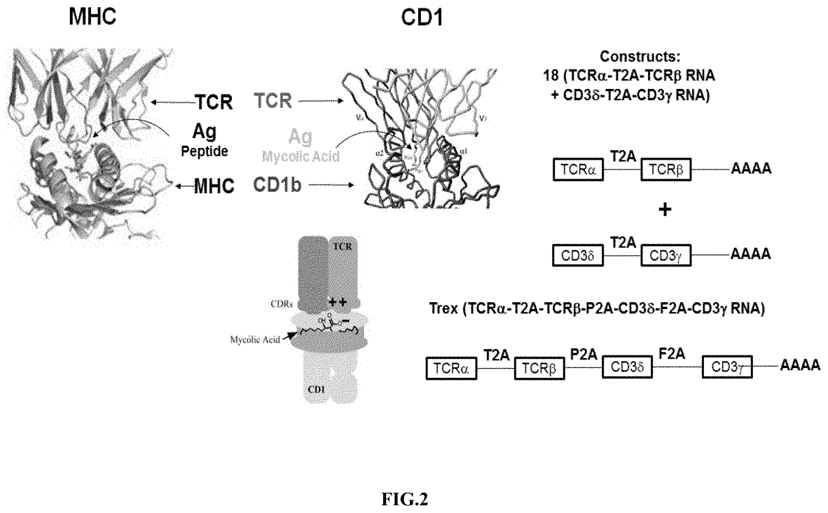

Additionally or alternatively, the inventors contemplate that cytotoxic immune competent cells (e.g., NK cells, genetically engineered NK cells, NKT cells, etc.) can also be genetically modified by introducing a recombinant nucleic acid composition encoding a protein complex to the cytotoxic cells. As shown in FIG. 2, and in an especially preferred embodiment, the protein complex includes at least one or more distinct peptides having an extracellular domain of a T cell receptor, and at least one or more distinct peptide of the intracellular domain of T cell co-receptor. For example, one preferred protein complex includes a T cell receptor α chain, a T cell receptor β chain, at least a portion of CD3δ, and at least a portion of CD3γ. In another example, the protein complex may include a γ chain T cell receptor and a δ chain T cell receptor instead of the α and β chains of T cell receptors. Additionally, or alternatively, the protein complex may also include one or more ζ-chains (which may be native to the cytotoxic cell or recombinant). Such nucleic acids may be isolated from clone 18 of T cell clone (clone 18) that recognizes free mycolic acid, a deglycosylated form of GMM (glucose-6-O-monomycolate) (see e.g., Nature Communications volume 7, Article number: 13257 (2016); Nat Immunol. 2013 July; 14(7): 706-713; or RCSB PDB Entry 4G8E).

Thus, it should be noted that where the recombinant cytotoxic cell is an NK cell, theT cell receptor alpha and beta chains can be expressed from a recombinant nucleic acid (preferably in a monocistronic or polycistronic mRNA) to form a functional T cell receptor with (a) the CD3 zeta and CD3 epsilon portions that are natively expressed in NK cells and (b) with the CD3 delta and Gamma portions that will be expressed from a recombinant nucleic acid (again, preferably in a monocistronic or polycistronic mRNA). FIG. 2 depicts two exemplary mRNA constructs that encode separately (a) the TCR alpha and beta chain and (b) CD3 delta and CD3 gamma. In yet another aspect of the inventive subject matter, all four recombinant components may also be expressed from a single mRNA construct (Trex) that encodes the TCR alpha and beta chain and CD3 delta and CD3 gamma in a molecule.

While any suitable forms of recombinant nucleic acid composition to encode the protein complex can be used, the inventors contemplate that the protein complex can be encoded by a single nucleic acid comprising a plurality of segments, each of which encodes a distinct peptide. Thus, in one preferred embodiment, the nucleic acid composition includes a first nucleic acid segment encoding two distinct peptides: an α chain T cell receptor and a β chain T cell receptor (or alternatively, γ chain T cell receptor and δ chain T cell receptor), and a second nucleic acid segment encoding two peptides: at least a portion of one type of T-cell co-receptor (e.g., CD3δ) and at least a portion of another type of T-cell co-receptor (e.g., CD3γ), or alternatively, encoding one or more -chain substituting for the portion of CD3δ or the portion of CD3γ. It is contemplated that each distinct peptide encoded by the first and second nucleic acid segments is a full length protein (e.g., full length alpha and β chain T cell receptor and co-receptors). Yet, it is also contemplated that at least one or more distinct peptides encoded by the first and second nucleic acid segments can be a truncated or a portion of the full length proteins.

Preferably, in one embodiment (18A/B as shown in FIG. 2), the first and second nucleic acid segments are mRNAs, each of which comprises two sub-segments of mRNA, which encode T cell receptor (e.g., sub-segment A is an mRNA of α chain T cell receptor and sub-segment B is an mRNA of β chain T cell receptor, etc.), followed by poly A tail. It is further preferred that the two sub-segments of mRNA are separated by nucleic acid sequences encoding a type of 2A self-cleaving peptide (2A). As used herein, 2A self-cleaving peptide (2A) refers any peptide sequences that can provide a translational effect known as “stop-go” or “stop-carry” such that two sub-segments in the same mRNA fragments can be translated into two separate and distinct peptides. Any suitable types of 2A peptide sequences are contemplated, including porcine teschovirus-1 2A (P2A), thosea asigna virus 2A (T2A), equine rhinitis A virus 2A (E2A), foot and mouth disease virus 2A (F2A), cytoplasmic polyhedrosis virus (BmCPV 2A), and flacherie virus (BmIFV 2A). In some embodiments, same type of 2A sequence can be used between two sub-segments of both first and second nucleic acid segments (e.g., fist nucleic acid segment: mRNA of a chain receptor—T2A—mRNA of β chain receptor; second nucleic acid segment: mRNA of α chain receptor—T2A—mRNA of β chain receptor). In other embodiments, different types of 2A sequence can be used between two sub-segments of both first and second nucleic acid segments (e.g., fist nucleic acid segment: mRNA of α chain receptor—T2A—mRNA of β chain receptor; second nucleic acid segment: mRNA of α chain receptor—P2A—mRNA of β chain receptor).

Additionally, the inventors contemplate that the first and second nucleic acid segments can also be present in a single nucleic acid (mRNA), for example, connected by a 2A sequence. In this embodiment (Trex, as shown in FIG. 2), the sub-segments of first and second nucleic acid segments can be arranged in any suitable order (e.g., α chain-βchain-CD3γ-CD3δ, β chain-CD3γ-α chain-CD3δ, etc.), with any suitable combination of same of different 2A sequences (e.g., α chain-T2A-β chain-P2A-CD3γ-F2A-CD3δ, β chain-P2A-CD3γ-T2A-α chain-F2A-CD3δ, etc.), followed by poly A tail at the 3′ of the single mRNA.

With respect to the mRNA sequence of first and second nucleic acid segments, it is preferred that the mRNA sequences are selected based on the type of target cells, antigens, and/or the cells that will express the first and second nucleic acid segments. For example, it is preferred that the peptide encoded by the first nucleic acid segment has an actual or predicted affinity to CD1-lipid antigen complex at least with a KD of at least equal or less than 10⁻⁶M, preferably at least equal or less than 10⁻⁷M, more preferably at least equal or less than 10⁻⁸M. Any suitable methods to identify the first nucleic acid segment sequence that has high binding affinity to the respective CD1-lipid antigen complex are contemplated. For example, a nucleic acid sequence of first nucleic acid segment can be identified via a mass screening of peptides having various affinities to the CD1-lipid antigen complex via any suitable in vitro assays (e.g., flow cytometry, SPR assay, a kinetic exclusion assay, etc.).

The recombinant nucleic acids (either encoding the recombinant protein or the protein complex as described) are introduced into immune competent cells, preferably cytotoxic immune cells, more preferably NK cells, NK (or NK92) cell derivatives or NKT cells by any suitable means. Preferably, the recombinant nucleic acid can be inserted into a suitable vector to be introduced to and expressed in the cytotoxic immune cells. The suitable vector includes, but not limited to, any mammalian cell expression vector and a viral vector, depending on the methodology of introducing the recombinant nucleic acid to the cells. Alternatively, where the recombinant nucleic acid(s) is/are RNA, the nucleic acid may be transfected into the cells. It should also be recognized that the manner of recombinant expression is not limited to a particular technology so long as the modified cells are capable of producing the chimeric protein in a constitutive or inducible manner. Therefore, the cells may be transfected with linear DNA, circular DNA, linear RNA, a DNA or RNA virus harboring a sequence element encoding the chimeric protein, etc. Viewed form a different perspective, transfection may be performed via ballistic methods, virus-mediated methods, electroporation, laser poration, lipofection, genome editing, liposome or polymer-mediated transfection, fusion with vesicles carrying recombinant nucleic acid, etc.

For example, transfection may be performed using a nanoparticle comprising poly (beta-amino ester). It is contemplated that the nanoparticle is suitable to carry a plurality of mRNA molecules (the recombinant nucleic acid encoding the recombinant T cell receptor, or its transcript, etc.) as a cargo within the nanoparticle, as exemplarily shown in Moffett et al., Nature Communications, volume 8, Article number: 389 (2017), which is incorporated by reference herein. In some embodiments, the nanoparticle is a naked nanoparticle (e.g., without a targeting domain, etc.). In other embodiments, the nanoparticle may include a targeting domain (e.g., an antibody, an scFv, etc.) that binds to a cell specific molecule (e.g., CD3, CD4, etc.) for targeted delivery of the recombinant nucleic acid to specific types of immune cells.

Thus, it should also be appreciated that the recombinant nucleic acid may be integrated into the genome (via genome editing or retroviral transfection) or may be present as a stable or transient extrachromosomal unit (which may have replicating capability). For example, the recombinant nucleic acid that is used to transfect the cytotoxic cell may be configured as a viral nucleic acid and suitable viruses to transfect the cells include adenoviruses, lentiviruses, adeno-associated viruses, parvoviruses, togaviruses, poxviruses, herpes viruses, etc. Alternatively, the recombinant nucleic acid may also be configured as extrachromosomal unit (e.g., as plasmid, yeast artificial chromosome, etc), or as a construct suitable for genome editing (e.g., suitable for CRiPR/Cas9, Talen, zinc-finger nuclease mediated integration), or may be configured for simple transfection (e.g., as RNA, DNA (synthetic or produced in vitro), PNA, etc.). Therefore, it should also be noted that the cells may be transfected in vitro or in vivo.

With respect to recombinant viruses, it is contemplated that all known manners of making recombinant viruses are deemed suitable for use herein, however, especially preferred viruses include adenoviruses, adeno-associated viruses, alphaviruses, herpes viruses, lentiviruses, etc. Among other appropriate choices, adenoviruses are particularly preferred. Moreover, it is further generally preferred that the virus is a replication deficient and non-immunogenic virus, which is typically accomplished by targeted deletion of selected viral proteins (e.g., E1, E3 proteins). Such desirable properties may be further enhanced by deleting E2b gene function, and high titers of recombinant viruses can be achieved using genetically modified human 293 cells as has been recently reported (e.g., J Virol. 1998 February; 72(2): 926-933). Most typically, the desired nucleic acid sequences (for expression from virus infected cells) are under the control of appropriate regulatory elements well known in the art.

Without wishing to be bound by any specific theory, the inventors contemplate that the expression of the recombinant protein in the cytotoxic cells (e.g., NK cells, NKT cells, etc.) augments an immune response by adding a cytotoxicity-mediated immune response against the cells infected by the microorganism or against the cells expressing immunogenic self-lipids. More specifically, when the NK cell expresses the recombinant protein, specific recognition and/or high-affinity binding of extracellular single-chain variant fragment to a CD1-lipid antigen complex (e.g., CD1b-mycolic acid by M. tuberculosis infection) triggers the signaling cascade via the intracellular activation domain including Src-family kinase-mediated tyrosine phosphorylation of the ITAM sequence, followed by binding of tyrosine kinases Syk and ZAP70 to the ITAM and series of phosphorylation on the adaptor molecules by the tyrosine kinases. Viewed from a different perspective, a T cell-type adaptive immune response may be engineered into NK cells to so render the NK cells cytotoxic with high specificity to cells carrying the CD1-lipophilic ligand complex. Such reaction is especially advantageous for treatment of cells infected with M. tuberculosis as the NK cells not only lyse the infected cells, but also exhibit antimicrobial effect due to the granulysin present in NK cells.

The inventors also contemplate that the so genetically engineered cytotoxic cells can be administered to a patient that is infected with microorganism, having a tumor, or suffering from autoimmune diseases (so long as such cells of the patient present a lipid antigen in association with CD1). It is contemplated that the genetically engineered NK cells can be formulated in any pharmaceutically acceptable carrier (e.g., preferably formulated as a sterile injectable composition) with a cell titer of at least 1×10³ cells/ml, preferably at least 1×10⁵ cells/ml, more preferably at least 1×10⁶ cells/ml, and at least 1 ml, preferably at least 5 ml, more preferably and at least 20 ml per dosage unit. However, alternative formulations are also deemed suitable for use herein, and all known routes and modes of administration are contemplated herein. As used herein, the term “administering” genetically engineered cytotoxic cells refers to both direct and indirect administration of the genetically engineered cytotoxic cell formulation, wherein direct administration of the genetically engineered cytotoxic cells is typically performed by a health care professional (e.g., physician, nurse, etc.), and wherein indirect administration includes a step of providing or making available the genetically engineered cytotoxic cell formulation to the health care professional for direct administration (e.g., via injection, etc.).

In some embodiments, the genetically engineered cytotoxic cell formulation is administered via systemic injection including subcutaneous, subdermal injection, or intravenous injection. In other embodiments, where the systemic injection may not be efficient (e.g., for brain tumors, etc.), it is contemplated that the genetically engineered cytotoxic cell formulation is administered via intratumoral injection.

With respect to dose of the genetically engineered cytotoxic cell formulation administration, it is contemplated that the dose may vary depending on the status of infection by microorganism, types of microorganism (e.g., progression, severity, etc.), status of autoimmune disease, symptoms, tumor type, size, location, patient's health status (e.g., including age, gender, etc.), and any other relevant conditions. While it may vary, the dose and schedule may be selected and regulated so that the genetically engineered cytotoxic cell does not provide any significant toxic effect to the host normal cells, yet sufficient to be effective to induce an cytotoxic effect against infected cells or the tumor such that the number of infected microorganism is decreased, infected cells are killed/removed, and/or size of the tumor cells is decrease, etc.

With respect to the schedule of administration, it is contemplated that it may also vary depending on the status of infection by microorganism, types of microorganism, status of autoimmune disease, symptoms, tumor type, size, location, patient's health status (e.g., including age, gender, etc.), and any other relevant conditions. In some embodiments, a single dose of genetically engineered cytotoxic cell formulation can be administered at least once a day or twice a day (half dose per administration) for at least a day, at least 3 days, at least a week, at least 2 weeks, at least a month, or any other desired schedule. In other embodiments, the dose of the genetically engineered cytotoxic cell formulation can be gradually increased during the schedule, or gradually decreased during the schedule. In still other embodiments, several series of administration of genetically engineered cytotoxic cell formulation can be separated by an interval (e.g., one administration each for 3 consecutive days and one administration each for another 3 consecutive days with an interval of 7 days, etc.).

In some embodiments, the administration of the genetically engineered cytotoxic cell formulation can be in two or more different stages: a priming administration and a boost administration. It is contemplated that the dose of the priming administration is higher than the following boost administrations (e.g., at least 20%, preferably at least 40%, more preferably at least 60%). Yet, it is also contemplated that the dose for priming administration is lower than the following boost administrations. Additionally, where there is a plurality of boost administration, each boost administration has different dose (e.g., increasing dose, decreasing dose, etc.).

In some embodiments, the dose and schedule of the genetically engineered cytotoxic cell formulation administration may be fine-tuned and informed by cellular changes of the infected cells or cancer cells. For example, after a cancer patient is administered with one or more dose of genetically engineered cytotoxic cell formulation, a small biopsy of the cancer tissue is obtained in order to assess any changes (e.g., upregulation of NKG2D ligand, apoptosis rate, etc.) resulted from the stress induced by genetically engineered cytotoxic cell formulation. The assessment of cellular changes can be performed by any suitable types of technology, including immunohisto-chemical methods (e.g., fluorescence labeling, in-situ hybridization, etc.), biochemical methods (e.g., quantification of proteins, identification of post-translational modification, etc.), or omics analysis. Based on the result of the assessment, the dose and/or schedule of the genetically engineered cytotoxic cell formulations can be modified (e.g., lower dose if severe cytotoxicity is observed, etc.).

### Examples

Based on the above and further considerations, the inventors therefore contemplated that NK cells expressing the protein complex of a chain T cell receptor, a β chain T cell receptor, at least a portion of CD3δ, and at least a portion of CD3γ, increase the cell-mediated cytotoxicity specifically to the cells presenting CD1-lipid antigen complex, particularly where the alpha and beta chains of the T cell receptor recognize a lipid CD1 complex. To that end, the inventors cloned the alpha and beta chains of the clone 18 TCR (see PDB entry 4G8E) and CD3delta and CD3gamma into an expressible mRNA construct essentially as depicted in FIG. 2.

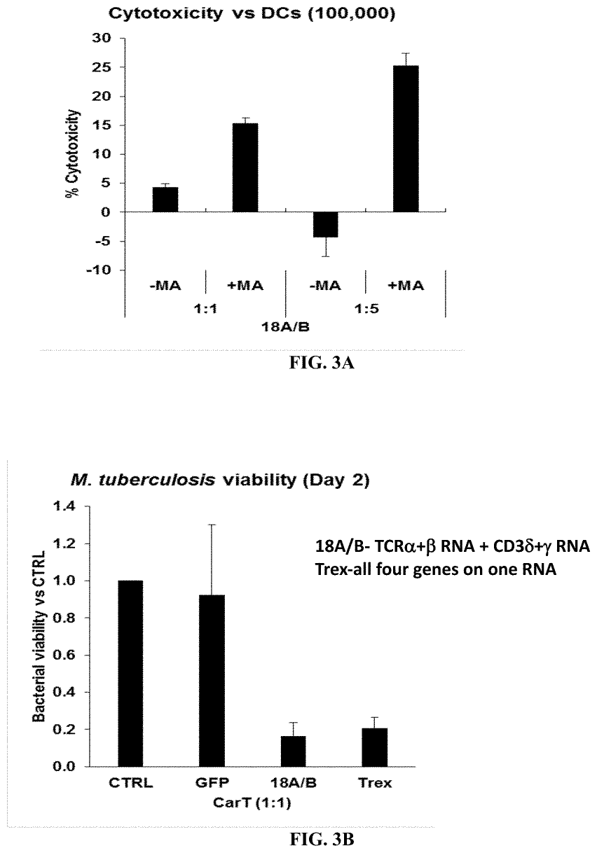

FIG. 3A shows one set of exemplary results in which recombinant NK cells expressing a recombinant T cell receptor as described above were incubated with dendritic cells expressing CD1d. More specifically, NK cells were genetically engineered to include two distinct and separate nucleic acid segments (18A/B): a first nucleic acid segment encoding two distinct peptides (an α chain T cell receptor and a β chain T cell receptor) and a second nucleic acid segment encoding two peptides (at least a portion of CD3δ and at least a portion of CD3γ). The cytotoxicity of the genetically engineered NK cells with 18A/B was then determined in four different conditions: with or without mycolic acid as a lipid antigen, and two different NK cell: dendritic cell (antigen presenting cell) ratios (1:1 and 1:5). The inventors found that the cytotoxicity of NK cells to the infected cells is specifically and significantly increased (by at least 3-5 times when the mycolic acids are present as a lipid antigen), confirming that the genetically modified NK cells can effectively function as hybrid cells that recognize the CD1-lipid antigen complex like a T cell, and elicit cytotoxicity to the cells presenting the CD1-lipid antigen complex as NK cytotoxic cells.

The inventors further found that NK cells that are genetically engineered to include two distinct and separate nucleic acid segments (18A/B) can produce cytotoxic effect against cells infected with M. tuberculosis as effective as NK cells that are that are genetically engineered to include a single nucleic acid segment (Trex) encoding all four components of the protein complex. As shown in FIG. 3B, NK cells expressing either 18A/B construct or Trex construct could kill about 70-80% of intracellular M. tuberculosis in the infected cells, which, in other words, reduces the M. tuberculosis viability to at least 20-25% in 2 days.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

1. A recombinant nucleic acid, comprising:
a first nucleic acid segment encoding an extracellular single-chain variant fragment that specifically binds to a CD1-lipid antigen complex;
a second nucleic acid segment encoding an intracellular activation domain;
a third nucleic acid segment encoding a linker between the extracellular single-chain variant fragment and the intracellular activation domain; and
wherein the first, second, and third segments are arranged such that the extracellular single-chain variant fragment, the intracellular activation domain, and the linker form a single chimeric polypeptide.

2. The recombinant nucleic acid of claim 1, wherein the extracellular single-chain variant fragment comprises a VL domain and a VH domain of a monoclonal antibody against the CD1-lipid antigen complex.

3. The recombinant nucleic acid of claim 2, wherein the extracellular single-chain variant fragment further comprises a spacer between the VL domain and the VH domain.

4. The recombinant nucleic acid of claim 1, wherein the CD1-lipid antigen complex comprises at least one of the following: CD1a, CD1b, CD1c.

5. The recombinant nucleic acid of claim 1, wherein the CD1-lipid antigen complex comprises at least one of the following: mycobacterial phospholipids, glycolipids, mycolic acids, lipopeptides, mycoketides, and isoprenoids.

6. The recombinant nucleic acid of claim 5, wherein the CD1-lipid antigen complex comprises a lipid antigen of M. tuberculosis.

7. The recombinant nucleic acid of claim 6, wherein the lipid antigen of M. tuberculosis is a mycolic acid.

8. The recombinant nucleic acid of claim 1, wherein the intracellular activation domain n comprises an immunoreceptor tyrosine-based activation motif (ITAM) that triggers ITAM-mediated signaling in a natural killer cell.

9. The recombinant nucleic acid of claim 1, wherein the intracellular activation domain comprises a portion of CD3ζ.

10. The recombinant nucleic acid of claim 1, wherein the intracellular activation domain further comprises a portion of CD28 activation domain.

11. The recombinant nucleic acid of claim 1, wherein the linker comprises a CD28 transmembrane domain or a CD3ζ transmembrane domain.

12. A recombinant nucleic acid composition, comprising:
a first nucleic acid segment encoding an α chain T cell receptor and a β chain T cell receptor, the α and β chain receptor being separated by a first self-cleaving 2A peptide sequence;
a second nucleic acid segment encoding at least a portion of CD3δ and at least a portion of CD3γ, the at least portion of CD3δ and the at least portion of CD3γ being separated by a second self-cleaving 2A peptide sequence; and
wherein at least one of the α chain T cell receptor and the β chain T cell receptor together specifically bind a CD1-lipid antigen complex.

13. The recombinant nucleic acid composition of claim 12, wherein the first nucleic acid segment and the second nucleic acid segment are separated by a third self-cleaving 2A peptide sequence.

14. The recombinant nucleic acid composition of claim 12, wherein the portion of CD3γ comprises an ITAM.

15. The recombinant nucleic acid composition of claim 12, wherein the portion of CD3δ comprises an ITAM.

16. The recombinant nucleic acid composition of claim 12, wherein the CD1-lipid antigen complex comprises at least one of the following: CD1a, CD1b, CD1c.

17. The recombinant nucleic acid of claim 12, wherein the CD1-lipid antigen complex comprises at least one of the following: mycobacterial phospholipids, glycolipids, mycolic acids, lipopeptides, mycoketides, and isoprenoids.

18. The recombinant nucleic acid of claim 17, wherein the CD1-lipid antigen complex comprises a lipid antigen of M. tuberculosis.

19. The recombinant nucleic acid of claim 18, wherein the lipid antigen of M. tuberculosis is a mycolic acid.

20-42. (canceled)

43. A genetically modified cytotoxic cell, comprising a recombinant nucleic acid encoding a chimeric protein having 1) an extracellular single-chain variant fragment that specifically binds a CD1-lipid antigen complex, 2) an intracellular activation domain, and 3) a transmembrane linker coupling the extracellular single-chain variant fragment to the intracellular activation domain.

44-65. (canceled)
